(19) United States
(12) Patent Application Publication
(10) Pub. No.: US 2015/0336995 A1
(43) Pub. Date: Nov. 26, 2015

(54) PROCESS FOR PREPARING 2,2'-BIPHENOLS USING SELENIUM DIOXIDE AND HALOGENATED SOLVENT

(71) Applicants: Katrin Marie DYBALLA, Recklinghausen (DE); Robert Farnke, Marl (DE); Dirk Fridag, Halterna am See (DE); Siegfried R. Waldvogel, Gau-Algesheim (DE); Thomas Quell, Mainz (DE); Michael Mirion, Mainz (DE)

(72) Inventors: Katrin Marie DYBALLA, Recklinghausen (DE); Robert Farnke, Marl (DE); Dirk Fridag, Halterna am See (DE); Siegfried R. Waldvogel, Gau-Algesheim (DE); Thomas Quell, Mainz (DE); Michael Mirion, Mainz (DE)

(73) Assignee: EVONIK DEGUSSA GmbH, Essen (DE)

(21) Appl. No.: 14/719,003

(22) Filed: May 21, 2015

(30) Foreign Application Priority Data
May 26, 2014 (DE) 102014209976.5

(51) Int. Cl. C07F 11/00; C07C 37/11

(52) U.S. Cl. CPC C07F 11/00; C07C 37/11

(57) ABSTRACT

2,2′-biphenol is prepared in a process using selenium dioxide and a halogenated solvent.

### BACKGROUND OF THE INVENTION

1. Field of the Invention

The invention relates to a process for preparing 2,2′-biphenol using selenium dioxide and a halogenated solvent.

2. Discussion of the Background

The direct coupling of phenols to give the corresponding biphenol derivatives which are of great industrial interest continues to be a challenge since these reactions are often neither regio- nor chemoselective.

The term “phenols” is used as a generic term in this application and therefore also encompasses substituted phenols.

One possible way of synthesizing these biphenols is by means of electrochemical processes. In this case, carbon electrodes such as graphite, glassy carbon, BDD or transition metals such as platinum are used (cf. F. Stecker, A. Fischer, I. M. Malkowsky, S. R. Waldvogel, A. Kirste, WO 2010139687 A1 and A. Fischer, I. M. Malkowsky, F. Stecker, S. R. Waldvogel, A. Kirste WO 2010023258 A1). A disadvantage of these electrochemical methods is the cost of some of the apparatus, which has to be manufactured specially. Moreover, scale-up to the ton scale, as is typically required in industry, is sometimes very complex and in some cases even impossible.

Direct cross-coupling of unprotected phenol derivatives under conventional organic conditions has been possible only in a few examples to date. For this purpose, usually superstoichiometric amounts of inorganic oxidizing agents such as AlCl₃, FeCl₃, MnO₂, or DDQ, which is organic, are used (cf. G. Sartori, R. Maggi, F. Bigi, M. Grandi, J. Org. Chem. 1993, 58, 7271).

Alternatively, such coupling reactions are conducted in a multistage sequence. In this case, leaving functionalities and often toxic, conjugated transition metal catalysts based on palladium, for example, are used.

A great disadvantage of the abovementioned methods for phenol coupling is the need for dry solvents and for exclusion of air. Both mean a high level of complexity, specifically when the process is to be used on the industrial scale.

Furthermore, the reactions described in the related art often give rise to toxic by-products which have to be removed from the desired product in a complex manner and disposed of at great cost. The increasing scarcity of raw materials (for example boron and bromine) and the rising relevance of environmental protection is increasing the cost of such transformations. Particularly in the case of utilization of multistage syntheses, an exchange of various solvents is necessary, which constitutes a high level of complexity and is an additional cost factor.

### SUMMARY OF THE INVENTION

It was an object of the invention to provide a process which does not have the disadvantages described in connection with the related art. More particularly, a process by which 2,2′-biphenols can be prepared selectively is to be provided, i.e. one in which the preparation gives rise to a minimum amount of by-products. The process should also be usable on the industrial scale.

This and other objects have been achieved by the present invention which provides a process for preparing a 2,2′-biphenol, comprising:

a) adding a first phenol to a reaction mixture,

b) adding a second phenol to the reaction mixture,

c) adding selenium dioxide to the reaction mixture,

d) adding a fluorinated solvent or a chlorinated solvent to the reaction mixture,

e) heating the reaction mixture such that the first phenol and the second phenol are converted to the 2,2′-biphenol.

### DETAILED DESCRIPTION OF THE INVENTION

The process for preparing 2,2′-biphenol, comprises the process steps of:

a) adding a first phenol to the reaction mixture,

b) adding a second phenol to the reaction mixture,

c) adding selenium dioxide to the reaction mixture,

d) adding a fluorinated solvent or a chlorinated solvent to the reaction mixture,

e) heating the reaction mixture such that the first phenol and the second phenol are converted to a 2,2′-biphenol.

Steps a) to d) can be conducted here in any sequence.

Any ranges mentioned herein include all values and sub-values between the upper and lower limits of the range.

A problem with the use of selenium dioxide is that the corresponding 2,2′-selenobiaryl ether and the corresponding Pummerer ketone can be obtained as by-products in large amounts. In the case of an unfavourable reaction regime, it may even be the case that the 2,2′-selenobiaryl ether is the main product of the reaction. According to the objective of the invention, the aim, however, is to conduct the reaction specifically in such a way that the level of such by-products is reduced to a minimum.

Through addition of selenium dioxide as oxidizing agent, depending on the reaction conditions, 2,2′-biphenols or 2,2′-selenobiaryl ethers can be obtained as main products of the reaction (cf. Scheme 1).

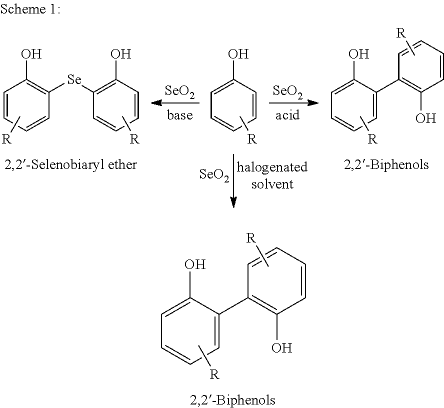

It has been found that the reaction can be shifted in the direction of the product desired in each case through addition of a base or an acid or a halogenated solvent.

In the present case, the 2,2′-biphenol is the desired main product.

Further advantages over the processes described in the related art are that it is not necessary to work with exclusion of moisture or oxygen. This constitutes a distinct advantage over other synthesis routes. This direct method of C—C coupling is an efficient and selective process which stands out advantageously from the existing multistage synthesis routes.

As a result of predominant formation of the desired main product and reduction in the formation of higher molecular weight overoxidation products, the workup is distinctly simplified.

Unconverted reactants and solvents used can be recovered by distillation and used for further reactions. Thus, the process according to the invention fulfils the requirements for an economic industrial scale process.

Moreover, selenium dioxide is used in the process according to the invention. Selenium dioxide is a waste product from metal purification and ore refining. Thus, in the process claimed here, a waste product from other processes is reused with addition of value. This is an important topic especially against the background of the sustainability of processes.

In one variant of the process, the first phenol in process step a) is a compound of the general formula I:

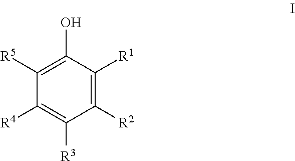

where R¹, R², R³, R⁴, R⁵ are each independently selected from the group consisting of:

—H, —(C₁-C₁₂)-alkyl, —O—(C₁-C₁₂)-alkyl, —(C₆-C₂₀)-aryl, —O—(C₆-C₂₀)-aryl, -halogen (such as Cl, F, Br, I), —OC═O—(C₁-C₁₂)-alkyl,

two adjacent radicals may additionally be joined to one another to form a condensed system,

where the alkyl and aryl groups mentioned may be substituted,

and at least R¹ or R⁵ is —H.

(C₁-C₁₂)-Alkyl and O—(C₁-C₁₂)-alkyl may each be unsubstituted or substituted by one or more identical or different radicals selected from the group consisting of:

(C₃-C₁₂)-cycloalkyl, (C₃-C₁₂)-heterocycloalkyl, (C₆-C₂₀)-aryl, fluorine, chlorine, cyano, formyl, acyl or alkoxycarbonyl.

(C₆-C₂₀)-Aryl and O—(C₆-C₂₀)-aryl may each be unsubstituted or substituted by one or more identical or different radicals selected from:

—H, —(C₁-C₁₂)-alkyl, —O—(C₁-C₁₂)-alkyl, —O—(C₆-C₂₀)-aryl, —(C₆-C₂₀)-aryl, -halogen (such as Cl, F, Br, I), —COO—(C₁-C₁₂)-alkyl, —CONH—(C₁-C₁₂)-alkyl, —(C₆-C₂₀)-aryl-CON[(C₁-C₁₂)-alkyl]₂, —CO—(C₁-C₁₂)-alkyl, —CO—(C₆-C₂₀)-aryl, —COOH, —OH, —SO₃H, —SO₃Na, —NO₂, —CN, —NH₂, —N[(C₁-C₁₂)-alkyl]₂.

In the context of the invention, the expression (C₁-C₁₂)-alkyl encompasses straight-chain and branched alkyl groups. Preferably, these groups are unsubstituted straight-chain or branched (C₁-C₈)-alkyl groups and most preferably (C₁-C₆)-alkyl groups. Examples of (C₁-C₁₂)-alkyl groups are especially methyl, ethyl, propyl, isopropyl, n-butyl, isobutyl, sec-butyl, tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,1-dimethylbutyl, 1,2-dimethylbutyl, 2,2-dimethylbutyl, 1,3-dimethylbutyl, 2,3-dimethylbutyl, 3,3-dimethylbutyl, 1,1,2-trimethylpropyl, 1,2,2-trimethylpropyl, 1-ethylbutyl, 1-ethyl-2-methylpropyl, n-heptyl, 2-heptyl, 3-heptyl, 2-ethylpentyl, 1-propylbutyl, n-octyl, 2-ethylhexyl, 2-propylheptyl, nonyl, decyl.

The elucidations relating to the expression —(C₁-C₁₂)-alkyl also apply to the alkyl groups in —O—(C₁-C₁₂)-alkyl, i.e. in —(C₁-C₁₂)-alkoxy. Preferably, these groups are unsubstituted straight-chain or branched —(C₁-C₆)-alkoxy groups.

Substituted (C₁-C₁₂)-alkyl groups and substituted (C₁-C₁₂)-alkoxy groups may have one or more substituents, depending on their chain length. The substituents are preferably each independently selected from:

—(C₃-C₁₂)-cycloalkyl, —(C₃-C₁₂)-heterocycloalkyl, —(C₆-C₂₀)-aryl, fluorine, chlorine, cyano, formyl, acyl or alkoxycarbonyl.

In one variant of the process, R¹, R², R³, R⁴, R⁵ are each independently selected from:

—H, —(C₁-C₁₂)-alkyl, —O—(C₁-C₁₂)-alkyl, —(C₆-C₂₀)-aryl, —O—(C₆-C₂₀)-aryl,

where the alkyl and aryl groups mentioned may be substituted,

and at least R¹ or R⁵ is —H.

In one variant of the process, R¹, R², R³, R⁴, R⁵ are each independently selected from:

—H, —(C₁-C₁₂)-alkyl,

where the alkyl and aryl groups mentioned may be substituted,

and at least R¹ or R⁵ is —H.

In one variant of the process, R¹, R³, R⁵ are each independently selected from:

—H, —(C₁-C₁₂)-alkyl,

where the alkyl groups mentioned may be substituted,

and at least R¹ or R⁵ is —H.

In one variant of the process, R² and R⁴ are each —H.

In one variant of the process, the second phenol in process step b) is a compound of the general formula II:

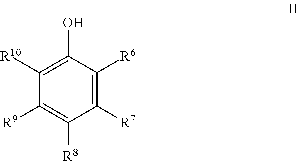

where R⁶, R⁷, R⁸, R⁹, R¹⁰ are each independently selected from:

—H, —(C₁-C₁₂)-alkyl, —O—(C₁-C₁₂)-alkyl, —(C₆-C₂₀)-aryl, —O—(C₆-C₂₀)-aryl, -halogen (such as Cl, F, Br, I), —OC═O—(C₁-C₁₂)-alkyl,

two adjacent radicals may additionally be joined to one another to form a condensed system,

where the alkyl and aryl groups mentioned may be substituted,

and at least R⁶ or R¹⁰ is —H.

In one variant of the process, R⁶, R⁷, R⁸, R⁹, R¹⁰ are each independently selected from:

—H, —(C₁-C₁₂)-alkyl, —O—(C₁-C₁₂)-alkyl, —(C₆-C₂₀)-aryl, —O—(C₆-C₂₀)-aryl,

where the alkyl and aryl groups mentioned may be substituted,

and at least R⁶ or R¹⁰ is —H.

In one variant of the process, R⁶, R⁷, R⁸, R⁹, R¹⁰ are each independently selected from:

—H, —(C₁-C₁₂)-alkyl,

where the alkyl and aryl groups mentioned may be substituted,

and at least R⁶ or R¹⁰ is —H.

In one variant of the process, R⁶, R⁸, R¹⁰ are each independently selected from:

—H, —(C₁-C₁₂)-alkyl,

where the alkyl groups mentioned may be substituted,

and at least R⁶ or R¹⁰ is —H.

In one variant of the process, R⁷ and R⁹ are each —H.

In one variant of the process, the first phenol corresponds to the second phenol.

This variant is thus a homo-coupling of two identical phenols. Ortho-ortho coupling thus gives rise to the desired 2,2′-biphenols.

In one variant of the process, the selenium dioxide is added in process step c) in a molar ratio based on the sum total of the first and second phenols within a range from 0.25 to 1.2.

Preference is given here to the range from 0.25 to 0.9, and particular preference to the range from 0.4 to 0.7.

The fact that the selenium dioxide can be used in a substoichiometric amount is a further advantage over the reaction described in the related art with other inorganic oxidizing agents, for example AlCl₃, FeCl₃ or MnO₂.

In one variant of the process, a fluorinated solvent is added in process step d).

In one variant of the process, a fluorinated carboxylic acid or a fluorinated alcohol is added as solvent in process step d).

In one variant of the process, trifluoroacetic acid or 1,1,1,3,3,3-hexafluoro-2-propanol is added as solvent in process step d).

In one variant of the process, the reaction mixture is heated in process step e) to a temperature in the range from 50° C. to 110° C.

Preference is given here to the range from 60° C. to 100° C., and particular preference to the range from 70° C. to 90° C.

The temperatures specified here are the temperatures measured in the oil bath.

In one variant of the process, the heating is effected in process step e) over a period in the range from 5 minutes to 24 hours.

Preference is given here to the range from 15 minutes to 2.5 hours, and particular preference to the range from 15 minutes to 2.0 hours.

Having generally described this invention, a further understanding can be obtained by reference to certain specific examples which are provided herein for purposes of illustration only, and are not intended to be limiting unless otherwise specified.

### EXAMPLES

Analysis

NMR Spectroscopy

The mass spectroscopy studies were conducted on multi-nucleus resonance spectrometers of the AC 300 or AV II 400 type from Bruker, Analytische Messtechnik, Karlsruhe. The solvent used was CDCl₃. The ¹H and ¹³C spectra were calibrated according to the residual content of undeuterated solvent using the NMR Solvent Data Chart from Cambridge Isotopes Laboratories, USA. Some of the ¹H and ¹³C signals were assigned with the aid of H,H-COSY, H,H-NOESY, H,C-HSQC and H,C-HMBC spectra. The chemical shifts are reported as δ values in ppm. For the multiplicities of the NMR signals, the following abbreviations were used: s (singlet), bs (broad singlet), d (doublet), t (triplet), q (quartet), m (multiplet), dd (doublet of doublets), dt (doublet of triplets), tq (triplet of quartets). All coupling constants J were reported in hertz (Hz) together with the number of bonds covered. The numbering given in the assignment of signals corresponds to the numbering shown in the formula schemes, which do not necessarily have to correspond to IUPAC nomenclature.

General Procedure

8.2 mmol of the particular phenol are dissolved in the appropriate solvent (8.2 M). The reaction mixture is heated, and 4.9 mmol of selenium dioxide are added while stirring. The solvent is distilled under reduced pressure (temperature <70° C.). A frit is prepared with 2.5 cm of silica gel (at the bottom) and 2.5 cm of zeolite (at the top). The distillation residue is taken up in the eluent and applied to the filtration column. Cyclohexane:ethyl acetate (95:5) is used to wash the product off the fit and collect it in fractions. The fractions containing product are combined and freed of the eluent by distillation.

The fractions obtained are recrystallized from 95:5 cyclohexane:ethyl acetate. For this purpose, the solid residue is dissolved at 50° C., and insoluble residues are filtered off using a glass frit. The reaction product crystallizes out of the saturated solution at room temperature overnight. The resulting crystals are washed once again with cold cyclohexane.

The structural formula shows the main product obtained in each reaction.

### 3,3′,5,5′-Tetramethylbiphenyl-2,2′-diol

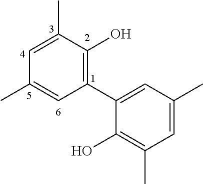

The reaction is conducted according to the general procedure in a screw-top test tube. For this purpose, 1.00 g (8.2 mmol, 1.0 equiv.) of 2,4-dimethylphenol and 0.54 g (4.9 mmol, 0.6 equiv.) of selenium dioxide are dissolved and heated in 1 ml of acid. The product is obtained as a beige crystalline solid.

¹H NMR (300 MHz, CDCl₃):

δ (ppm)=7.00 (s,2H, 6-H), 6.87 (s, 2H, 4-H), 5.07 (s,2H, OH), 2.27 (s, 12H, 3-CH₃, 5-CH₃).

¹³C NMR (75 MHz, CDCl₃):

δ (ppm)=149.2 (C-2), 132.1 (C-4), 130.0 (C-5), 128.5 (C-6), 125.1 (C-3), 122.1 (C-1), 20.4 (5-CH₃), 16.2 (3-CH₃).

### Bis(3, 5-dimethyl-2-hydroxyphenyl)selenium

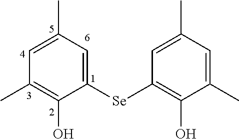

The reaction is conducted according to the general procedure in a screw-top test tube. For this purpose, 1.00 g (8.2 mmol, 1.0 equiv.) of 2,4-dimethylphenol and 0.54 g (4.9 mmol, 0.6 equiv.) of selenium dioxide are dissolved and heated in 1 ml of pyridine. The product is obtained as a colourless crystalline solid.

¹H NMR (400 MHz, CDCl₃):

δ (ppm)=7.12 (s,2H, 6-H), 6.91 (s, 2H, 4-H), 5.97 (s,2H, OH), 2.23 (s, 6H, 3-CH₃) 2.23 (s, 6H, 5-CH₃).

¹³C NMR (100 MHz, CDCl₃):

δ (ppm)=151.7 (C-2),133.2 (C-3), 133.1 (C-5), 130.4 (C-4), 124.2 (C-6), 114.9 (C-1), 20.3 (5-CH₃), 16.5 (3-CH₃).

⁷⁷Se NMR (76 MHz, CDCl₃):

δ (ppm)=163.36 ppm.

### 3,3′,5,5′-Tetra-tert-butylbiphenyl-2,2′-diol

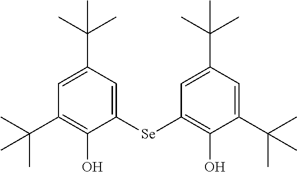 [SMILES: CC(C)(C)c1cc([Se]c2cc(C(C)(C)C)cc(C(C)(C)C)c2O)c(O)c(C(C)(C)C)c1]

The reaction is conducted according to the general procedure in a screw-top test tube. For that purpose, 1.67 g (8.2 mmol, 1.0 equiv.) of 2,4-di-tert-butylphenol and 0.55 g (4.9 mmol, 0.6 equiv.) of selenium dioxide were dissolved and heated in 1 ml of pyridine.

¹H NMR (400 MHz, CDCl₃):

δ (ppm)=7.31 (d, J=2.4 Hz, 2H), 7.29 (d, J=2.4), 6.29 (s, 2H), 1.42 (s, 18H), 1.24 (s, 18H).

¹³C NMR (75 MHz, CDCl₃):

δ (ppm)=151.7, 143.5, 135.8, 129.8, 125.6, 117.2, 35.4, 34.4, 31.6, 29.7.

### Bis(3,5-Di-tert-butyl-2-hydroxyphenyl)selenium

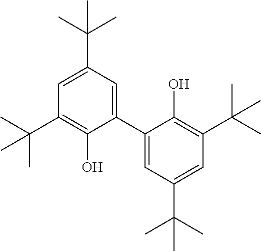 [SMILES: CC(C)(C)c1cc(-c2cc(C(C)(C)C)cc(C(C)(C)C)c2O)c(O)c(C(C)(C)C)c1]

The reaction is conducted according to the general procedure in a screw-top test tube. For that purpose, 307 mg (1.5 mmol, 1.0 equiv.) of 2,4-di-tert-butylphenol and 99 mg (0.8 mmol, 0.6 equiv.) of selenium dioxide were dissolved and heated in 0.5 ml of acetic acid.

¹H NMR (400 MHz, CDCl₃):

δ (ppm)=7.39 (d, J=2.4 Hz, 2H), 7.11 (d, J=2.4, 2H), 5.21 (s, 2H), 1.45 (s, 18H), 1.32 (s, 18H).

¹³C NMR (75 MHz, CDCl₃):

δ (ppm)=149.9, 143.0, 125.4, 124.9, 122.4, 35.4, 34.6, 31.7, 29.8.

The results of the above-described reaction, and variations thereof, are shown in the tables which follow. The processes according to the invention are identified here by *.

The following compound classes are specified in detail in the tables:

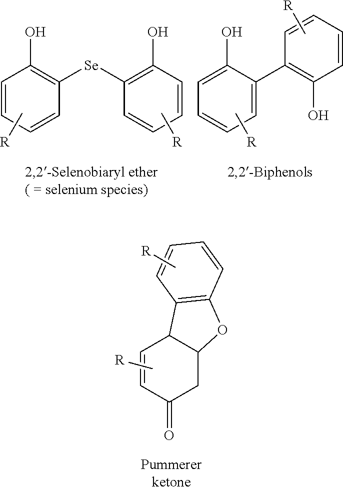

TABLE 1a
Oxidative coupling of 2,4-dimethylphenol
Basic conditions

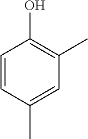

| | | | | | | Selenium |
|---|---|---|---|---|---|---|
| | T | t | | Pummerer | Biphenol | species |
| Solvent | [° C.] | [h] | pKb | ketone [%] | [%] | [%] |
| Pyridine | 60 | 5 | 8.9 | — | — | 79.1 |
| Pyridine | 85 | 5 | 8.9 | 2.6 | 13.1 | 59.6 |
| Pyridine | 100 | 0.5 | 8.9 | 1.9 | 11.0 | 39.9 |
| Triethylamine | 80 | 4 | 3.3 | — | — | 1.8 |
| (dry) | | | | | | |
| DMF | 85 | 5 | −1.1 | 4.2 | 19.1 | 18.8 |

It can be inferred from Table 1a that (with the exception of dimethylformamide (DMF)), the desired biphenol is obtained only as a by-product. In the case of DMF, the biphenol and the unwanted selenium species form in about equal portions.

TABLE 1b
Oxidative coupling of 2,4-dimethylphenol
Acidic conditions

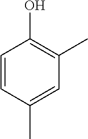

| | | | | | | Selenium |
|---|---|---|---|---|---|---|
| | T | t | | Pummerer | Biphenol | species |
| Solvent | [° C.] | [h] | pKa | ketone [%] | [%] | [%] |
| Acetic acid | 85 | 5 | 4.8 | 4.5 | 74.8 | 1.98 |
| Acetic acid | 60 | 1.5 | 4.8 | 1.8 | 39.8 | 8.0 |
| Methane- | 85 | 5 | −2.6 | — | 3.9 | 6.1 |
| sulphonic | | | | | | |
| acid | | | | | | |
| p- | 85 | 5 | −2.8 | — | 15.0 | — |
| Toluene- | | | | | | |
| sulphonic | | | | | | |
| acid | | | | | | |

It can be inferred from Table 1b that, when acetic acid was used, it was possible to prepare the biphenol in a good yield in each case. Using methanesulphonic acid and p-toluenesulphonic acid, it was possible to obtain the desired biphenol only in very low yields.

TABLE 1c
Oxidative coupling of 2,4-dimethylphenol
Halogenated solvent

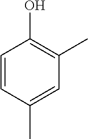

| | | | | | | Selenium |
|---|---|---|---|---|---|---|
| | T | t | | Pummerer | Biphenol | species |
| Solvent | [° C.] | [h] | pKa | ketone [%] | [%] | [%] |
| HFIP* | 60 | 2 | 9.2 | — | 90.3 | 0.6 |
| Trifluoroacetic | 85 | 5 | 0.23/ | 2.5 | 77.8 | 1.4 |
| acid*/acetic acid | | | 4.8 | | | |
| (3:1) | | | | | | |

HFIP: 1,1,1,3,3,3-hexafluoro-2-propanol

It is clear from Table 1c that the yields of biphenol listed in Table 1b could actually be exceeded through the use of HFIP as solvent. Through the addition of three parts trifluoroacetic acid to one part acetic acid, it was also possible to slightly increase the yield of biphenol, and distinctly reduce the proportion of Pummerer ketone.

TABLE 2a
Oxidative coupling of 2,4-di-tert-butylphenol
Basic conditions

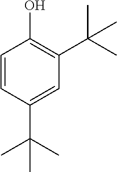

| | T | t | | Biphenol | Selenium species |
|---|---|---|---|---|---|
| Solvent | [° C.] | [h] | pKb | [%] | [%] |
| Pyridine | 40 | 24 | 8.9 | 20.6 | 46.8 |
| Pyridine | 60 | 7 | 8.9 | 10.1 | 30.4 |

Under basic conditions, the selenium species again forms as the main product of the reaction.

TABLE 2b
Oxidative coupling of 2,4-di-tert-butylphenol
Acidic conditions

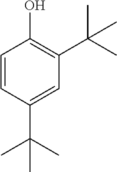

| | T | t | | Biphenol | Selenium species |
|---|---|---|---|---|---|
| Solvent | [° C.] | [h] | pKa | [%] | [%] |
| Acetic acid | 50 | 18 | 4.8 | 29.5 | 25.2 |
| Acetic acid | 85 | 1 | 4.8 | 25.9 | 23.1 |

Under acidic conditions, the biphenol is the main product of the reaction; it was obtained in a slight excess compared to the selenium species.

TABLE 2c
Oxidative coupling of 2,4-di-tert-butylphenol
Halogenated solvent

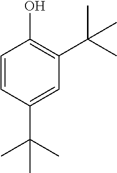

| | T | t | | Biphenol | Selenium species |
|---|---|---|---|---|---|
| Solvent | [° C.] | [h] | pKa | [%] | [%] |
| HFIP* | 60 | 1 | 9.2 | 42.9 | 12.8 |

HFIP: 1,1,1,3,3,3-hexafluoro-2-propanol

Under the conditions according to the invention, the desired biphenol is the main product of the reaction. It can be obtained in a distinct excess compared to the selenium species.

The results summarized in Tables 1a to 2c show clearly that the process according to the invention fulfils the objective defined above. The process according to the invention is a synthesis route by which 2,2′-biphenols can be prepared selectively, in a good yield. In addition, the process according to the invention can also be implemented on the industrial scale.

Further comparative tests are described hereinafter.

2,4-Dimethylphenol was reacted with SeO₂.

TABLE 3
Overview of the reaction of 2,4-dimethylphenol with selenium
dioxide with various temperatures and solvents after
18 hours. 1.3 equivalents of selenium dioxide (based
on 2,4-dimethylphenol) were used in each case.

| | T | t | 2,4-Dimethyl | Pummerer | | Selenium |
|---|---|---|---|---|---|---|
| Solvent | [° C.] | [h] | phenol | ketone | Biphenol | species |
| THF | 90 | 18 | 98 | 1 | 1 | — |
| Glyme | 96 | 18 | — | 17 | 74 | — |
| Glyme | 75 | 18 | 13 | 9 | 52 | 10 |
| Diglyme | 96 | 18 | 95 | 1 | 4 | — |

If glyme (ethylene glycol dimethyl ether) is used as solvent, the biphenol is obtained as the main product here too. However, high temperatures and long reaction times are required, which makes the reaction unattractive for industrial scale use. A further disadvantage is that it is necessary to use the selenium dioxide in superstoichiometric amounts, i.e. in more than 1.0 equivalent. Furthermore, the sum total of the by-products (Pummerer ketone+selenium species) is above 15% in each case. The high proportion of secondary components makes the corresponding workup to obtain the pure substance much more difficult and hence also costlier, which is disadvantageous for an industrial scale process.

With tetrahydrofuran (THF) (pKb 11.5) as solvent, hardly any biphenol forms.

Using the process according to the invention, much better reaction results can be achieved in a much shorter time.

German patent application 102014209976.5 filed May 26, 2014, is incorporated herein by reference.

Numerous modifications and variations on the present invention are possible in light of the above teachings. It is therefore to be understood that within the scope of the appended claims, the invention may be practiced otherwise than as specifically described herein.

1. A process for preparing a 2,2′-biphenol, comprising:
a) adding a first phenol to a reaction mixture,
b) adding a second phenol to the reaction mixture,
c) adding selenium dioxide to the reaction mixture,
d) adding a fluorinated solvent or a chlorinated solvent to the reaction mixture,
e) heating the reaction mixture such that the first phenol and the second phenol are converted to the 2,2′-biphenol.

2. The process according to claim 1, wherein the first phenol in process step a) is a compound of the general formula I: 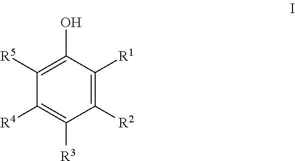
wherein R¹, R², R³, R⁴, R⁵ are each independently selected from the group consisting of:
—H, —(C₁-C₁₂)-alkyl, —O—(C₁-C₁₂)-alkyl, —(C₆-C₂₀)-aryl, —O—(C₆-C₂₀)-aryl, -halogen, and —OC═O—(C₁-C₁₂)-alkyl,
two adjacent radicals are optionally joined to one another to form a condensed system,
wherein the alkyl and aryl groups mentioned are optionally substituted,
and at least R¹ or R⁵ is —H.

3. The process according to claim 2, wherein R¹, R², R³, R⁴, R⁵ are each independently selected from the group consisting of:
—H, —(C₁-C₁₂)-alkyl, —O—(C₁-C₁₂)-alkyl, —(C₆-C₂₀)-aryl, and —O—(C₆-C₂₀)-aryl,
wherein the alkyl and aryl groups mentioned are optionally substituted,
and at least R¹ or R⁵ is —H.

4. The process according to claim 2, wherein R¹, R³, R⁵ are each independently selected from the group consisting of:
—H, and —(C₁-C₁₂)-alkyl,
wherein the alkyl groups mentioned are optionally substituted,
and at least R¹ or R⁵ is —H.

5. The process according to claim 1, wherein the second phenol in process step b) is a compound of the general formula II: 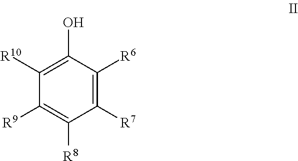
wherein R⁶, R⁷, R⁸, R⁹, R¹⁰ are each independently selected from the group consisting of:
—H, —(C₁-C₁₂)-alkyl, —O—(C₁-C₁₂)-alkyl, —(C₆-C₂₀)-aryl, —O—(C₆-C₂₀)-aryl, -halogen, and —OC═O—(C₁-C₁₂)-alkyl,
two adjacent radicals are optionally joined to one another to form a condensed system,
wherein the alkyl and aryl groups mentioned are optionally substituted,
and at least R⁶ or R¹⁰ is —H.

6. The process according to claim 5, wherein R⁶, R⁷, R⁸, R⁹, R¹⁰ are each independently selected from the group consisting of:
—H, —(C₁-C₁₂)-alkyl, —O—(C₁-C₁₂)-alkyl, —(C₆-C₂₀)-aryl, and —O—(C₆-C₂₀)-aryl,
wherein the alkyl and aryl groups mentioned are optionally substituted,
and at least R⁶ or R¹⁰ is —H.

7. The process according to claim 5, wherein R⁶, R⁸, R¹⁰ are each independently selected from the group consisting of:
—H, and —(C₁-C₁₂)-alkyl,
wherein the alkyl groups mentioned are optionally substituted,
and at least R⁶ or R¹⁰ is —H.

8. The process according to claim 1, wherein the first phenol is the same as the second phenol.

9. The process according to claim 1, wherein the selenium dioxide is added in process step c) in a molar ratio from 0.25 to 1.2, based on the sum total of the first and second phenols.

10. The process according to claim 1, wherein a fluorinated solvent is added in process step d).

11. The process according to claim 1, wherein a fluorinated carboxylic acid or a fluorinated alcohol is added as solvent in process step d).

12. The process according to claim 1, wherein trifluoroacetic acid or 1,1,1,3,3,3-hexafluoro-2-propanol is added as solvent in process step d).

13. The process according to claim 1, wherein the reaction mixture is heated in process step e) to a temperature in the range from 50° C. to 110° C.

14. The process according to claim 1, wherein the heating in process step e) is effected over a period in the range from 5 minutes to 24 hours.
